(19) United States
(12) Patent Application Publication
(10) Pub. No.: US 2025/0291070 A1
(43) Pub. Date: Sep. 18, 2025

(54) ESTIMATING A RATE OF RANDOMLY-OCCURRING COINCIDENCES IN A COUNTING X-RAY DETECTOR

(71) Applicant: Siemens Healthineers AG, Forchheim (DE)

(72) Inventors: Bodo REITZ, Forchheim (DE); Bjoern KREISLER, Hausen (DE); Michael HOSEMANN, Erlangen (DE); Edgar GOEDERER, Forchheim (DE); Martin HUPFER, Erlangen (DE); Sebastian KONRAD, Forchheim (DE)

(73) Assignee: Siemens Healthineers AG, Forchheim (DE)

(21) Appl. No.: 19/077,235

(22) Filed: Mar. 12, 2025

(30) Foreign Application Priority Data
Mar. 14, 2024 (EP) 24163525.9

(51) Int. Cl. G01T 1/172; G06T 7/00

(52) U.S. Cl. CPC G01T 1/172; G06T 7/0012

(57) ABSTRACT

A method for estimating a rate of randomly-occurring coincidences in a counting X-ray detector having a number of detector elements, the method comprising acquiring X-ray signals by the X-ray detector and converting the X-ray signals into electrical signals at the detector elements; passing on at least some of the electrical signals to signal inputs of a coincidence unit; counting coincidences of the signals passed on into the coincidence unit to determine at least one counting rate of acquired randomly-occurring coincidences; and estimating a rate of randomly-occurring coincidences based on the at least one determined counting rate.

### CROSS-REFERENCE TO RELATED APPLICATION(S)

The present application claims priority under 35 U.S.C. § 119 to European Patent Application No. 24163525.9, filed Mar. 14, 2024, the entire contents of which is incorporated herein by reference.

### FIELD

At least some example embodiments relate to a method for estimating a rate of randomly-occurring coincidences in a counting X-ray detector, a method for estimating a real coincidence, a method for recording an X-ray image dataset, a counting X-ray detector and a medical imaging device.

Independent of the grammatical term usage, individuals with male, female or other gender identities are included within the term.

### RELATED ART

Photon-counting X-ray detectors are used for many different applications, in particular, for imaging applications. By way of example, photon-counting detectors are used increasingly for deployment in computed tomography (CT) systems. Photon-counting X-ray detectors are typically based upon the principle that incident X-ray signals and/or X-ray photons are converted by a converter into electrical signals which can then be registered and evaluated. X-ray detectors with adjustable energy thresholds can be used, which enable an energy-resolving acquisition of the X-ray signals.

In order, firstly, to achieve a high spatial resolution and, secondly, to restrict the counting rates in the individual detector elements, the detector elements or pixel elements tend to be configured very small. However, this can have the disadvantage that the entire energy of an X-ray quantum is often not deposited in a single pixel, but is distributed over two or more-typically adjacent-detector elements and/or pixels, since the charge clouds generated in the detector spread over more than one detector element. It can therefore occur that photons are counted multiple times in adjacent detector elements. In this way, both the spatial resolution and also the energy resolution of the detector system can be restricted. The occurrence of these doublings can be designated coincidences and/or true coincidences or real coincidences.

An approach to solving this problem is to introduce, in addition to a counter for the counting rates of the acquired photons, a coincidence counter for each detector element which counts events in which at least one adjacent detector element is registered simultaneously with the detector element under consideration. The counting rates of the counters and coincidence counters can be acquired, in particular, for existing energy thresholds so that a coincidence counter counts events in which at least one adjacent pixel exceeds an energy threshold simultaneously with the pixel under consideration.

Approaches to the acquisition of the coincidences are known from the prior art and are described, for example, in DE 10 2012 224 209 A1, EP 3 839 577 A1 and EP 3 839 576 A1.

Such coincidence counters often have the tendency, however, to overestimate the abundance of the X-ray quanta from which the signal is distributed over a plurality of pixels. The reason for this is that the coincidence counter is incremented not only in the case of real coincidences but also when a second, independent X-ray quantum randomly deposits its energy in one of the adjacent pixels simultaneously with the X-ray quantum incident in the pixel under consideration—also known as randomly-occurring coincidences. On occurrence of randomly-occurring coincidences, the use of the coincidence counter can lead to an over-correction. Typically, the size of the error due to the non-observation of randomly-occurring coincidences increases strongly with higher photon fluxes. The reason for this is that the number of these events results, to a first approximation, from the product of the counting rate in the pixel, the counting rates in the adjacent pixel, the number of adjacent pixels and the length of the coincidence time window during which the real coincidences represent only a certain proportion of the counting rate in the pixel under consideration.

### SUMMARY

An estimation of the number of randomly-occurring coincidences from the individual counting rates of all pixels is possible to only a limited extent since, for this, the exact proportion of the real coincidences in the individual counting rates would have to be known, which however is typically not the case in a CT system, since this proportion is itself spectrally dependent.

One or more example embodiments provides a possibility which can at least lessen the problem of an overestimation of real coincidence in counting X-ray detectors and/or can provide an improved estimation of a real coincidence.

This is achieved at least by a method according to claim 1, a method according to claim 7, a method according to claim 13, a counting X-ray detector according to claim 14 and a medical imaging device according to claim 15. Further features and advantages are disclosed in the dependent claims, the description and the attached drawings.

### BRIEF DESCRIPTION OF THE DRAWINGS

Embodiments will now be described making reference to the accompanying drawings.

FIG. 1 shows a flow diagram of a method for estimating a rate of randomly-occurring coincidences in a counting X-ray detector, comprising a large number of detector elements according to one embodiment of the invention,

FIG. 2 shows a flow diagram of a method for estimating a real coincidence of two X-ray signals acquired simultaneously according to a predetermined criterion on adjacent detector elements of a counting X-ray detector according to one embodiment of the invention,

FIG. 3 shows a flow diagram of a method for recording an X-ray image, in particular, a computed tomography image, of an object with an X-ray system, in particular, a computed tomography system, according to one embodiment of the invention,

FIG. 4 shows a circuit example of a detector element according to one embodiment of the invention,

FIG. 5 shows a circuit example of a detector element according to a further embodiment of the invention,

FIG. 6 shows a circuit example of a detector element according to a further embodiment of the invention,

FIG. 7 shows further embodiment of the invention of the embodiment shown in FIG. 6 with regard to possibilities for selecting the detector elements that are used for determining the coincidences,

FIG. 8 shows a further embodiment of the invention of the embodiment shown in FIG. 6 with regard to possibilities for selecting the detector elements that are used for determining the coincidences,

FIG. 9 shows a further embodiment of the invention of the embodiment shown in FIG. 6 with regard to possibilities for selecting the detector elements that are used for determining the coincidences,

FIG. 10 shows a further embodiment of the invention with regard to a possibility for selecting the detector elements that are used for determining the coincidences,

FIG. 11 shows a further embodiment of the invention with regard to a possibility for selecting the detector elements that are used for determining the coincidences,

FIG. 12 shows a circuit example of a detector element according to a further embodiment of the invention,

FIG. 13 shows different variants according to the invention of the embodiment shown in FIG. 12 with regard to possibilities for selecting the detector elements that are used for determining the coincidences,

FIG. 14 shows a further embodiment of the invention with regard to a possibility for selecting the detector elements that are used for determining the coincidences,

FIG. 15 shows a further embodiment of the invention with regard to a possibility for selecting the detector elements that are used for determining the coincidences,

FIG. 16 shows a further embodiment of the invention with regard to a possibility for selecting the detector elements that are used for determining the coincidences,

FIG. 17 shows a variant of the placement of the circuit for establishing the randomly-occurring coincidence according to one embodiment of the invention, and

FIG. 18 shows a computed tomography system according to one embodiment of the invention.

### DETAILED DESCRIPTION

According to an embodiment of the invention, a method is provided for estimating a rate of randomly-occurring coincidences in a counting X-ray detector. The X-ray detector comprises a large number of detector elements. The method comprises the following steps:
(a) acquiring X-ray signals by the X-ray detector and converting the X-ray signals into electrical signals at the detector elements;
(b) passing on at least some of the electrical signals to signal inputs of a coincidence unit, wherein the signal inputs comprise a first signal input and at least one further signal input,
wherein the signals for the first signal input are acquired in a first of the detector elements,
wherein the signals for the at least one further signal input are each acquired in another detector element not directly adjacent to the first of the detector elements and/or
wherein signals for the at least one further signal input or the signals for the first signal input in an electrical circuit are temporally offset by a defined time interval before they are passed on to the coincidence unit;
(c) counting coincidences of the signals passed on into the coincidence unit in order to determine at least one counting rate of acquired randomly-occurring coincidences;
(d) estimating a rate of randomly-occurring coincidences on the basis of the at least one determined counting rate.

Advantageously, the method according to one or more example embodiments of the invention provides a possibility for estimating randomly-occurring coincidences relatively reliably. This can enable, for example, the usable range for coincidence counters to be extended to higher X-ray flux levels in that, through knowledge of the randomly-occurring coincidences, the contributions of real coincidences and the randomly-occurring coincidences can be disentangled.

Randomly-occurring coincidences can also be designated independent coincidences or false coincidences or as random coincidences. The expression “randomly-occurring coincidences” is to be understood as distinct from the expression “real coincidences”. Randomly-occurring coincidences occur when two primary X-ray quanta randomly occur simultaneously in different detector elements, in particular, adjacent detector elements. Different factors, such as for example, the X-ray flux and the width of the detector elements can influence the rate of the randomly-occurring coincidences. Real coincidences can also be designated true coincidences. Real coincidences occur when the same event, that is in particular, a single X-ray quantum, triggers a signal in adjacent detector elements simultaneously. With counting X-ray detectors, the attempt is made to acquire the correct number of X-ray quanta and also their correct energy. Via the approach of acquiring coincidences, conclusions can be drawn regarding when individual photons and/or X-ray quanta trigger a plurality of signals so that the correct number of X-ray quanta can be better determined. However, this determination can be impaired by the randomly-occurring coincidences, so that an over-correction can be induced in that more real coincidences are counted than actually take place. Advantageously, by way of the method according to one or more example embodiments of the invention, randomly-occurring coincidences can be determined, so that consequently, in particular, in the event of large X-ray fluxes, the real coincidences can be more precisely established and/or corresponding counting rates can be corrected.

The counting X-ray detector can be, for example, a counting X-ray detector of a computed tomography system. The counting X-ray detector can also be designated a photon-counting X-ray detector. It is configured, in general, to acquire and count individual X-ray photons, broken down, in particular, spatially and/or temporally. The concept of X-ray signals is to be understood broadly in the context of this disclosure. Incident X-rays and/or X-ray photons can generally be designated X-ray signals. Typically, a counting X-ray detector comprises an X-ray converter in which incident X-rays generate mobile charge carriers, in particular, electron-hole pairs. Typically, electrical contacts are applied to the X-ray converter as electrodes to which a voltage is applied. Via the voltage, the charge carriers generated are transported to contacts and to readout electronics connected thereto, where they are typically amplified, the signal sizes are compared with thresholds and, on exceeding a defined threshold, are converted into electrical signals to be output, in particular, logical and/or digital electrical signals. In the context of this disclosure, electrical signals can be referred to as signals for short. The contacts together with at least parts of the connected readout electronics are part of the detector elements. The incoming signals can be counted by counters in the readout electronics. In counting X-ray detectors, in particular, in the context of computed tomography, typically a plurality of thresholds are used. Typically the detector elements have comparators with which it can be specified which minimum energy must be received so that a signal is counted. The principle of the use of threshold values and counters can be used both for counting incident photons in general and also for counting coincidences. Coincidence units can be provided for just one, for example the lowest, threshold or for a specific dedicated threshold. However, it is also conceivable for coincidence units to be provided for one or more thresholds or even for combinations of different thresholds.

The X-ray detector comprises a large number of detector elements. The detector elements can be arranged matrix-like. In particular, a plurality of detector elements can be associated with a partial detector. The detector elements can be evenly distributed. Optionally, the detector elements can be arranged group-wise. The detector elements can also be known with other designations such as pixels, pixel elements, image point elements, etc. In general, the term detector element is to be broadly interpreted in the context of the disclosure. Apart from the explicitly named features and the necessary functionality, the design of the detector elements can be selected relatively freely. Thus, for example, one, some or all of the comparators or counters can be part of a detector element or can be configured separately.

The coincidence unit can, for example, also be designated the coincidence circuit. In the context of this disclosure, this coincidence unit for determining randomly-occurring coincidences can also be designated a random coincidence unit. The coincidence unit has a plurality of signal inputs and is configured, in particular, to acquire simultaneously incoming signals as coincidences and to generate a corresponding count signal for counting the coincidences. The signal inputs are provided, in particular, as the input for the electrical signals generated by way of X-ray radiation. The electrical signals can be filtered on the basis of threshold values. For example, it can be provided that only electrical signals with a defined minimum strength are conducted to the first signal input and/or to the second signal input. Signals that are acquired by a first of the detector elements are fed to the first signal input. For example, the first of the detector elements can be that for which the rate of the randomly-occurring coincidences is acquired. Alternatively, the first of the detector elements can be one different from that for which the rate of the randomly-occurring coincidences is acquired. For example, the first of the detector elements can be a detector element that is arranged close to the detector element for which the rate of the randomly-occurring coincidences is acquired. “Close to” can signify, for example, the adjacent or the next-but-one detector element. In the context of this disclosure, adjacent can mean, in particular, that the one detector element is the closest detector element to the output detector element in a particular direction. In particular, no detector element is situated directly between two adjacent detector elements. In a rectangular matrix of detector elements, detector elements are also referred to as adjacent if they are obliquely adjacent. In other words, detector elements which do not follow sequentially along the rectangular sides, but rather follow one another obliquely to the rectangular sides are also adjacent. “Obliquely” in the context of this disclosure means, in particular, along a diagonal of the rectangular matrix.

According to an alternative, it can be provided that the signals of the first signal input or of the further signal inputs are delayed. This can be realized, in particular, by way of a delay in the electrical circuit. Preferably, the delay is greater than a signal processing time of the detector elements. By way of the delay, it can be precluded that the signals are triggered by a single photon. Preferably, the delay is selected to be so small that a change in the X-ray flux in the time period of the delay is statistically negligible. Since the temporal sequence of signals of a plurality of X-ray quanta is uncorrelated, the counting rate of the randomly-occurring coincidences does not change if a signal path is delayed, provided this delay is short in comparison with the sampling in an X-ray device, for example, in a computed tomography device. Since an approximately unchanging statistic over the time period of the delay can therefore be assumed, a good measure for the randomly-occurring coincidence can be established, wherein real coincidences can simultaneously be substantially excluded by the delay.

According to a further alternative, the signals for the first signal input and for the at least one further signal input originate from detector elements which are not mutually directly adjacent. Preferably, the signals of the further signal inputs can originate from detector elements that are each the next-but-one detector element relative to the first detector element. In other words, it can be provided that, in each case, exactly one other detector element is arranged between the first detector element and the detector elements for the further signal inputs. Since the detector elements are not directly adjacent, it can be largely excluded that signals acquired there originate from the same X-ray photon. It is advantageous in this variant that a delay circuit is not necessarily needed. The delays of a plurality of further signal inputs also do not need to be coordinated and aligned. The effort and/or the space required for the circuit can thus be reduced. A randomly-occurring coincidence can thus be realized via a spatial separation.

Optionally preferably, not more than nine, particularly preferably, not more than five signal inputs are provided for the coincidence unit. For example, most particularly preferably, exactly 5 or exactly 3 signal inputs can be provided. From the signal inputs, in particular, one signal input can be provided for signals from the detector element for which a randomly-occurring coincidence is to be determined, and the remaining signal inputs for signals from further detector elements. In some embodiments, it can be particularly preferable to provide not more than four, or more particularly preferably, exactly two signal inputs for the coincidence unit. In particular, it can be provided in these embodiments that the signals for the signal inputs do not all come from the detector element for which a randomly-occurring coincidence is to be determined, but from further detector elements. Therefore since the number of detector elements for the estimation is small, the occurrence of counted randomly-occurring coincidences can be kept low. The counter therefore increments less rapidly with the X-ray flux and can thus advantageously deliver information over a more extended flux range. It has been found that even with two signal inputs, a sufficiently good statistic for estimating the rate of the randomly-occurring coincidences can be generated.

Optionally, the two alternatives given can also be combined.

Acquired coincidences can then be counted with the coincidence units in order to determine at least one counting rate of acquired randomly-occurring coincidences. Therefore a number of the randomly-occurring coincidences can be explicitly measured.

On the basis of the counted randomly-occurring coincidences, the rate of randomly-occurring coincidences can be estimated. Generally, the estimation can be more accurate if more electrical signals are used. However, the circuit complexity can be lower if fewer electrical signals are taken into account. The rate of the randomly-occurring coincidences can be scaled according to the actual setup.

Optionally, it can be provided that not all the registered electrical signals are used for the counting of coincidences, even regardless of their strength. For example, purely by way of example, coincidences can be established for a selection of electrical signals. Thereby, a circuit complexity can be reduced. A use of fewer signals can be allowed for by way of statistical calculations in the estimation of the rate of the randomly-occurring coincidences.

According to one embodiment, the signals for the at least one further signal input are each acquired in another of the detector elements, in particular, a detector element adjacent to the first detector element, wherein signals for the at least one further signal input or the signals for the first signal input in the electrical circuit are temporally offset with a defined time interval before they are passed on to the coincidence unit. Preferably, the time interval exceeds maximum transit time differences in the analogue and/or digital signals, preferably both, in the electrical circuit. Advantageously, a real coincidence cannot increment this counter. For example, the time interval can be in a range from 50 ns to 10 μs, preferably 80 ns to 300 ns, more preferably 100 ns to 200 ns. Since transit time differences of X-ray detectors are typically below these times and typical integration times of computed tomography systems are typically significantly higher, these ranges are particularly favorable. A range of more than 100 ns is particularly suitable to achieve a differentiation from typical transit times and thus also to obtain real coincidences. With an upper limit of 300 ns, it is therein particularly suitable to keep an influence of a temporally changing X-ray flux, for example, due to a further-moving detector, negligibly small. A time interval of not more than 200 ns is, however, more favorable since due to this transit time, the circuit complexity can be further reduced and a necessary output of the X-ray detector can be lessened. The use of adjacent detector elements can therein be particularly advantageous since thereby a corresponding setup that is very similar or even effectively identical to the real coincidences can be created.

According to one embodiment, the signals for the at least one further signal input are also each acquired in the first of the detector elements, wherein signals for the at least one further signal input or the signals for the first signal input are temporally offset with a defined time interval in the electrical circuit before they are passed on to the coincidence unit. The time interval can correspond to that of the embodiment with different detector elements. In this embodiment, an advantage can be found in that an interconnection between different detector elements can be reduced.

According to one embodiment, a plurality of coincidence units is provided, of which at least one coincidence unit is associated with each subgroup of detector elements, in particular, a subgroup of spatially adjacently arranged detector elements, wherein the method is applied for each of the plurality of coincidence units, wherein with each of the at least one of the plurality of coincidence units, the rate of randomly-occurring coincidences is estimated for the detector elements of the associated subgroup.

According to one embodiment, the rate of randomly-occurring coincidences is estimated with the at least one coincidence unit for a detector element that is to be estimated, the electrical signal of which is not itself fed into the coincidence unit, wherein the signals for the first signal input and the signals for the at least one further signal input originate from detector elements that are each adjacent to the detector element to be estimated. Advantageously, the randomly-occurring coincidences can thus be derived from detector elements that are spatially closer to the detector element under consideration, so that spatial variations in the counting rates across the detector can affect the value for the randomly-occurring coincidences less. Preferably, there is exactly one further signal input of the coincidence unit and the signals for the first signal input and for the one further signal input originate, in particular, from a total of two adjacent detector elements. Preferably, the detector elements are arranged in a rectangular matrix and the total of two adjacent detector elements for the first signal input and for the one further signal input are arranged obliquely adjacent to the detector element to be estimated.

According to one embodiment, at least one coincidence unit is associated with a subgroup of detector elements, in particular, a subgroup of spatially adjacently arranged detector elements, wherein with the at least one coincidence unit, the rate of randomly-occurring coincidences is estimated for the detector elements of the associated subgroup. Through the use of a coincidence unit for an entire subgroup, a circuit complexity can be reduced. However, a plurality of coincidence units, for example 2 to 4 coincidence units can be provided for the subgroup. In particular, it can be provided that the circuit for measuring the randomly-occurring coincidences is constructed only for a portion of the detector elements. For example, a subgroup can comprise 2 to 200, preferably 4 to 100, particularly preferably 10 to 50 detector elements. The subgroup can be arranged, for example, rectangular, wherein the side lengths of the rectangle are defined by the number of the detector elements in the subgroup. For example, the side lengths of the rectangle can be in the range of 2 to 10 detector elements. A range of 2 to 10 detector elements in each direction can be particularly favorable, since in this range the counting rates typically do not differ too severely and, simultaneously, a good saving of circuit complexity is possible. Preferably, a coincidence unit is provided per 2 to 36 detector elements, particularly preferably a coincidence unit per 6 to 24 detector elements.

Preferably only one pair of detector elements is used for the determination of the randomly-occurring coincidences within a subgroup. In particular, one coincidence unit is preferably provided per subgroup, wherein the coincidence unit has exactly two signal inputs for signals from two detector elements. Preferably, 4 to 100, particularly preferably 9 to 36 detector elements can be provided per subgroup. The detector elements can be arranged in a rectangular matrix. The matrix can have the form N×M, wherein N and M are the number of the detector elements along the sides of the rectangular matrix. N and M can each have a value, for example, from 2 to 10, preferably 3 to 6. For example, the detector elements can be arranged in a rectangular 4×6 matrix. It has been found that with such a matrix in this embodiment, particularly good results can be achieved with simultaneously efficient savings of circuits. Preferably, the detector elements which are used for the signal inputs of the detector unit are arranged in the subgroup such that each other detector element is at least a next-but-one neighbor to one of these two detector elements.

According to one embodiment, the detector elements are divided into subgroups of detector elements, wherein intermediate spaces are available between the subgroups, wherein at least one coincidence unit, in particular, the coincidence unit or the plurality of coincidence units is or are arranged in the intermediate spaces between the subgroups. In particular, the at least one coincidence unit can be arranged in a region in the intermediate space that lies in the shadow of an anti-scatter grid. Advantageously, in this embodiment, the circuit for the coincidence unit can be displaced out of the typically restricted space directly under and/or on the detector elements into the region between the detector elements. The space directly under and/or on the detector elements is typically restricted since normally electronic circuits for the processing of the signals of the detector elements are arranged there. For example, it can be provided to configure the coincidence units of adjacently arranged subgroups respectively alternatingly on different sides, in particular, so that two coincidence units are always together in one intermediate space. This can be particularly advantageous, for example, for the further processing of the count signals of the randomly-occurring coincidences. This variant can be combined with other variants described herein, in particular, variants relating to subgroups. In particular, a variant with a pair of detector elements for the determination of the randomly-occurring coincidences within a subgroup can advantageously be combined with this embodiment.

A further example embodiment of the invention is a method for estimating a real coincidence of two X-ray signals acquired simultaneously according to a predetermined criterion on adjacent detector elements of a counting X-ray detector. The method comprises the following steps:
carrying out the method for estimating a rate of randomly-occurring coincidences as described herein; and
determining the coincidence of two X-ray signals acquired simultaneously according to the predetermined criterion on adjacent detector elements in order to establish uncorrected coincidences, in particular, in that for each detector element with at least one detector element adjacent thereto, optionally with each adjacent detector element, a number of coincidence count signals is counted;
correcting the uncorrected coincidences on the basis of the rate of randomly-occurring coincidences in order to estimate the real coincidence. The predetermined criterion can comprise, for example, a time period within which events are assessed as coincident and/or simultaneous. The real coincidence can be acquired and/or transferred, in particular, as a count value.

All the advantages and features of the method for estimating a rate of randomly-occurring coincidences in a counting X-ray detector can be transferred likewise to the method for estimating a real coincidence and vice versa. Advantageously, by way of the establishment, in particular, on the basis of an explicit measurement, the contribution of real coincidences and of those randomly occurring in coincidence counters can be separated. Thereby, for example, a usable measuring range for coincidence counters can be extended to significantly higher X-ray fluxes and thereby also the advantages of a coincidence circuit. A possible over-correction by way of excessively highly estimated real coincidences can be counteracted. In particular, this can be enabled without being reliant upon model assumptions or empirical corrections.

The determination of the coincidence of two X-ray signals acquired simultaneously according to the predetermined criterion on adjacent detector elements can be carried out, for example, in accordance with a method known from the prior art. For example, electrical signals acquired via a coincidence counter coincidently, i.e. simultaneously within a particular tolerance, can be registered and counted in adjacent detector elements. For the registration of the coincidences, for example, a coincidence unit can be provided which acquires cases in which an incoming electrical signal of a respective threshold of the detector element is received in a specified timeframe simultaneously with an electrical signal of a threshold from one or more adjacent detector elements. For this, both the electrical signal of this detector element and also electrical signals of the adjacent detector elements can be fed to the coincidence unit and/or to signal inputs of the coincidence unit. A coincidence counter can then count the number of the coincidence events in which at least one adjacent detector element exceeds a threshold simultaneously with the detector element under consideration.

According to one embodiment, the rate of randomly-occurring coincidences is estimated with the at least one coincidence unit for a detector element that is to be estimated, the electrical signal of which is not itself fed into the coincidence unit, wherein the signals for the first signal input and the signals for the at least one further signal input originate from detector elements that are each adjacent to the detector element to be estimated. Preferably, the detector elements from which the signals for the signal inputs originate are not adjacent to one another. Preferably, there is exactly one further signal input and the signals for the first signal input and for the one further signal input originate, in particular, from a total of two adjacent detector elements. Preferably, the detector elements are arranged in a rectangular matrix and the total of two adjacent detector elements for the first signal input and for the one further signal input are arranged obliquely adjacent to the detector element to be estimated. Preferably, the uncorrected coincidence is carried out on the basis of the detector element that is to be estimated itself and four detector elements adjacent thereto, wherein the four adjacent detector elements, in particular, are preferably not the two adjacent detector elements for the first signal input and for the one further signal input. In particular, the four detector elements which are used for the establishment of the uncorrected coincidence can be arranged along the rectangular sides of the rectangular matrix adjacent to the detector element to be estimated.

According to one embodiment, both for establishing the uncorrected coincidences and also for estimating the randomly-occurring coincidences, a coincidence unit is provided in each case, wherein for each of the two coincidence units the same number of signal inputs are provided for electrical signals the coincidences of which are being counted. This embodiment can enable a particularly simple adaptation of the uncorrected coincidences, in particular, since the rate of the randomly-occurring coincidences typically does not need to be further scaled.

According to one embodiment, the same detector elements are each provided for determining the uncorrected coincidence as well as for estimating the randomly-occurring coincidence, wherein in particular, the detector element the real coincidence of which is to be determined and its adjacent detector elements are provided. With this, a particularly accurate estimation of the randomly-occurring coincidence can be enabled. In particular, the signals of the detector element the real coincidence of which is to be determined, or the signals of the adjacent detector elements are delayed before they are fed to the coincidence unit for estimation of the randomly-occurring coincidences. The randomly-occurring coincidence can be determined on the basis of the temporal offset at a defined time interval. In other words, in this embodiment, the digital coincidence circuit for each detector element can be doubled and, for each detector element, two coincidence values can be counted. The uncorrected coincidence value is therein based upon the coincidence of the signal of a detector element and that of the detector elements adjacent thereto without a delay. This value represents, in particular, the sum of real and randomly-occurring coincidences. For the second signal path, either the signal of the detector element or the signal of all the adjacent detector elements can be delayed. This count value represents, in particular, the number of the randomly-occurring coincidences.

According to one embodiment, for determining the uncorrected coincidence and also for estimating the randomly-occurring coincidence, signals are provided for the signal inputs of the respective coincidence unit from the detector element, the real coincidence of which is to be determined, and from one or more of its adjacent detector elements, wherein only a subgroup of the adjacent detector elements is provided for determining the uncorrected coincidence and/or for estimating the randomly-occurring coincidence. According to one alternative embodiment, for determining the randomly-occurring coincidence, signals are provided for the signal inputs of the coincidence unit for estimating the randomly-occurring coincidences of the detector element, the real coincidence of which is to be determined, and of detector elements next-but-one thereto, wherein only a subgroup of the next-but-one detector elements is provided for determining the randomly-occurring coincidence and/or only a subgroup of the adjacent detector elements is provided for determining the uncorrected coincidence.

Since only one subgroup is taken into account, advantageously, the circuit complexity can be reduced. The subgroup can optionally consist of just one detector element.

In addition, the number of coincidences counted in this way can be reduced. Thereby, information regarding randomly-occurring coincidences can be estimated over a broader X-ray flux range. With the use of such a value based upon a subgroup for correcting the count value for estimating the real coincidences, the count value and/or the rate of the randomly-occurring coincidences can be scaled with the ratio of the number of adjacent detector elements which are used for the establishing of the uncorrected coincidences. Optionally, for the establishing of the uncorrected coincidences, a subgroup can also be used, for example, an equal-sized subgroup or a subgroup with a different number of detector elements. For example, obliquely adjacent detector elements typically contribute less to real coincidences and therefore often no substantial error is introduced if these are dispensed with. It can also be useful to restrict the circuit to fewer signal inputs of the respective coincidence unit at the edges of a detector.

According to one embodiment, the real coincidence is transferred as a count value, wherein in addition to the real coincidence, the randomly-occurring coincidence and/or the uncorrected coincidence is also transferred as an additional count value in each case. Also to transfer the randomly-occurring coincidence and/or the uncorrected coincidence can make it possible to use them in a further evaluation and/or for subsequent checking of the real coincidences. For example, the transferred count values of the randomly-occurring coincidences can be taken into account in a reconstruction computer, in an integrated circuit, in particular, an FPGA (field programmable gate array), in an internal infrastructure of the detector and/or in an external circuit infrastructure of the detector with other count values, for example, the uncorrected coincidences. Advantageously, therefore, on the basis, for example, of the actual count values, it can be decided, for example subsequently, which corrections are used. Optionally, a mixing degree of the corrections can be a function of one or more further parameters. Further parameters can be, for example, the counting rate, the number of the uncorrected or real coincidences, the number of the randomly-occurring coincidences and/or other, in particular, X-ray flux-dependent variables. For example, at very high X-ray fluxes, linear pile-up effects can accumulate. On the basis thereof, for example, on the basis of the mixing degree, a fluid transition from the full use of corrections to a discarding of the count values can be controlled. For example, the mixing degree can be a factor of 1 and in the case of a particularly large X-ray flux, a factor of 0. In an alternative embodiment, it can also be provided that only the uncorrected coincidence and the randomly-occurring coincidence are transferred. In this case, it can be provided, in particular, that the real coincidence is determined retrospectively. In a further alternative embodiment, it can also be provided that only the real coincidence is transferred and, in particular, not the randomly-occurring coincidence and not the uncorrected coincidence.

According to one embodiment, a counting rate of the randomly-occurring coincidences is used in order to monitor a paralysis of another counter of signals and possibly to correct it, in particular, a paralysis of another counter of signals the randomly-occurring coincidence of which is estimated with this counting rate. The other counter can be, for example, a counter for counting incident X-ray photons. In the event of excessively high X-ray fluxes, at least some counters can become paralyzed in that they are no longer able to acquire further photons. This can even have the result that, above a particular rate of incident photons, a counter registers lower values again. In this way, ambiguities can occur because it is not clear whether the counter is registering an actually low X-ray flux or whether the counter is paralyzed. The counter of the randomly-occurring coincidences is probably less often paralyzed since coincidences of events typically occur less often than single events. Counters, in particular, with a low energy threshold can be paralyzable, that is counters which register a majority of the incident photons. Such counters are designated paralyzable counters. Advantageously, the count signal of the random coincidence counter can be used in order to linearize a paralyzable counter so that, in particular, a monotonically rising non-paralyzable behavior can be achieved.

According to one embodiment, the correction of the uncorrected coincidences is carried out on the basis of the rate of randomly-occurring coincidences in the front end of the X-ray detector. This embodiment can be relatively simple to implement since only one simple difference in the count values must be generated. A scaling of the count values can be capable of implementation relatively easily in the form of simple multiplications. If an X-ray detector, for example, in a computed tomography system is subdivided into subgroups, there are typically some edges and corners in which the number of the contributing detector elements for both the real coincidences and also for the randomly-occurring coincidences is smaller than for the plurality of other detector elements. This difference can be taken into account in the scaling and possibly also by way of an implementation of different embodiments. Specifically for this case, the offsetting and/or correction in the front end can be particularly advantageous. For example, the exact wiring, i.e. the respective number of detector elements used, at each site can be known and taken into account in a targeted manner.

According to one embodiment, a pulser signal is mixed into at least some of the electrical signals converted by the detector elements. In particular, a signal with a mixed-in pulser signal is applied to not more than one of the signal inputs of a coincidence unit in each case. The pulser signal can be provided, for example, for calibrations, dead time measurements and/or for preventing a paralysis of the circuit at high X-ray fluxes. The pulser signal can be mixed in, for example, as a clock signal. By way of regular feeding-in of a clock signal, a signal can also be counted and thus a non-paralyzability can be created. Pulser signals are typically fully or partially correlated with one another across large detector ranges. Since a signal with a mixed-in pulser signal is applied to not more than one of the signal inputs of one coincidence unit in each case, the occurrence of systematic effects which can falsify the count value with artificially generated coincidences can be prevented.

A further example embodiment of the invention is a method for recording an X-ray image dataset, in particular, a computed tomography image dataset, of an object with an X-ray system, in particular, a computed tomography system with a counting X-ray detector. The X-ray detector comprises a large number of detector elements. The method comprises the following steps:
counting at least one number of count signals dependent upon the incident X-ray radiation in each detector element; and
carrying out a method for estimating a real coincidence as described herein;
generating an X-ray image dataset on the basis of the at least one number of count signals counted in each detector element and the estimated real coincidence.

All the advantages and features of the method for estimating a rate of randomly-occurring coincidences in a counting X-ray detector and of the method for estimating a real coincidence can be transferred likewise to the method for recording an X-ray image and vice versa. When the X-ray image dataset is created, image values, in particular, voxel values and/or pixel values on the basis of the at least one number of count signals and the estimated real coincidence can be provided. The number of count signals can be corrected and/or adapted by way of the real coincidence. In particular, an image reconstruction can be based upon the adapted at least one number of count signals. For example, a number of coincidence count signals can be subtracted from the at least one number of count signals. For example, a weighted subtraction can be provided, in particular, in which before the subtraction a mixing factor is multiplied by the number of coincidence count signals. This means that just a portion or a multiple of the at least one number of coincidence count signals can be subtracted or added. For the counting of at least one number of count signals, a threshold can be used so that signals below this threshold are not counted. If a plurality of numbers of count signals are counted for a detector element, then for each of the numbers, a threshold can be used, wherein in particular for the different numbers of a detector element, different thresholds can be used. The coincidences can be determined for one of the thresholds which are used for the number of count signals. For example, one of the thresholds can be the lowest of the thresholds. The use of only one threshold can advantageously reduce a circuit complexity. However, it is also conceivable to provide a plurality of thresholds for the coincidence units or for combinations of different thresholds. Preferably, for determining the uncorrected coincidence and the randomly-occurring coincidence, the same threshold and/or the same thresholds are used.

According to one embodiment, for the counting of the number of count signals in each detector element, only X-ray signals are acquired the energy of which exceeds a first minimum threshold, wherein for the determination of the randomly-occurring coincidence, only X-ray signals are acquired the energy of which exceeds a second minimum threshold, wherein the first minimum threshold and the second minimum threshold in particular differ. Consequently, a dedicated specific threshold can be used for the coincidence measurement.

A further example embodiment of the invention is a counting X-ray detector, in particular, for a computed tomography system, for recording an X-ray image dataset of an object transirradiated by an X-ray radiation, wherein the X-ray detector comprises a large number of detector elements and at least one electrical circuit with at least one coincidence unit, wherein the X-ray detector is configured to carry out a method as described herein. The at least one electrical circuit can be, at least partially, part of a detector element. For example, each detector element can comprise an electrical circuit. All the advantages and features of the method for estimating a rate of randomly-occurring coincidences in a counting X-ray detector, of the method for estimating a real coincidence and of the method for recording an X-ray image can be transferred likewise to the counting X-ray detector and vice versa. The X-ray detector can comprise an X-ray converter in which incident X-rays generate mobile charge carriers. The detector elements can be configured, in particular, to acquire the mobile charge carriers generated by the X-ray converter and further to process them as an electrical signal. The detector elements can each comprise at least one comparator. The comparator can comprise an adjustable signal threshold. The detector elements can comprise counters to count electrical signals, in particular, as described herein.

A example embodiment of the invention is a medical imaging device, in particular, a computed tomography system with a counting X-ray detector, in particular, an X-ray detector as described herein and a control module, wherein the medical imaging device, in particular, the computed tomography system is configured to carry out a method as described herein. In particular, the control module can be configured to control the execution of the method. The control module can be implemented, for example, in the form of a computer, a microcontroller or an integrated circuit or, in each case, a part thereof. The control module can comprise hardware elements and/or software elements. The control module can optionally be a grouping of computers or a cloud or, in each case, a part thereof. All the advantages and features of the method for estimating a rate of randomly-occurring coincidences in a counting X-ray detector, of the method for estimating a real coincidence, of the method for recording an X-ray image and of the counting X-ray detector can be transferred likewise to the computed tomography system and vice versa.

All the embodiments described herein can be combined with one another if not explicitly stated otherwise.

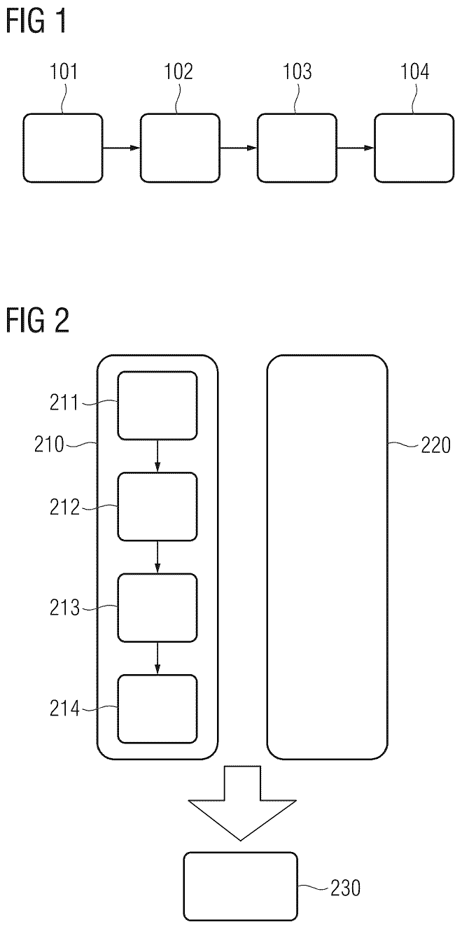

FIG. 1 shows a flow diagram of a method for estimating a rate of randomly-occurring coincidences in a counting X-ray detector 31, comprising a large number of detector elements according to one embodiment of the invention. In a first step 101, X-ray signals are acquired by the X-ray detector 31 and the X-ray signals are converted on the detector elements into electrical signals. This can take place, for example, through X-ray radiation being converted in an X-ray converter into mobile charge carriers and then by way of an applied voltage, fed into the detector element at the respective position and amplified there and compared with a threshold, so that when the threshold is exceeded, the electrical signals are output. In a fourth step 102, at least some of the electrical signals are passed on to signal inputs of a coincidence unit. The signal inputs comprise a first signal input and at least one further signal input. The signals for the first signal input are acquired in a first of the detector elements. The signals for the at least one further signal input are each acquired in another detector element not directly adjacent to the first of the detector elements. Since the signals for the at least one further signal input are acquired in a detector element that is not directly adjacent, it can be substantially ensured that the signals do not originate from the same X-ray photon as the signals for the first signal input. In addition or alternatively, the signals for the at least one further signal input or the signals for the first signal input in an electrical circuit are temporally offset by a defined time interval before they are passed on to the coincidence unit. By way of the temporal offset, it is also possible for the signals of the first signal input and of the at least one further signal input not to originate from the same X-ray photon. In a further step 103, coincidences of the signals passed on into the coincidence unit are counted in order to determine at least one counting rate of acquired randomly-occurring coincidences. On the basis of the counting rate, in a further step 104, a rate of randomly-occurring coincidences is estimated. The rate of randomly-occurring coincidences can result directly from the counting rate of acquired randomly-occurring coincidences. Alternatively, the counting rate can be adapted, in particular, scaled in order to estimate the rate of randomly-occurring coincidences.

FIG. 2 shows a flow diagram of a method for estimating a real coincidence of two X-ray signals acquired simultaneously according to a predetermined criterion on adjacent detector elements of a counting X-ray detector 31 according to one embodiment of the invention. In a higher-level step 210, a method for estimating a rate of randomly-occurring coincidences is carried out. The individual steps 211-214 of this higher-order step 210 can correspond, for example, to the steps 101-104 set out in relation to FIG. 1. In a further higher-level step 220, a method for determining the coincidence of two X-ray signals acquired simultaneously according to the predetermined criterion on adjacent detector elements is carried out. With this, uncorrected coincidences are established, in particular, in that for at least some detector elements with at least one detector element adjacent thereto, a number of coincidence count signals is counted. The number of coincidence count signals can be counted, for example, in each detector element or in a portion of the detector elements. The counting is based upon the directly incoming signals in the respective detector element and upon a coincidently occurring signal of at least one adjacent detector element. Preferably, these two steps 210, 220 are carried out substantially at the same time. In a further step 230, the uncorrected coincidences are corrected on the basis of the rate of randomly-occurring coincidences in order to estimate the real coincidence.

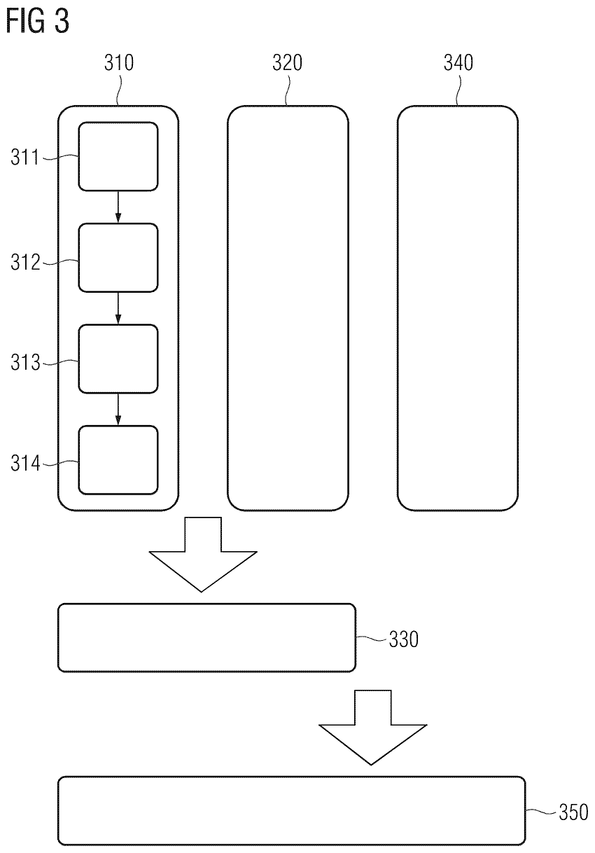

FIG. 3 shows a flow diagram of a method for recording an X-ray image, in particular, a computed tomography image, of an object with an X-ray system, in particular, a computed tomography system, according to one embodiment of the invention. The steps 310-330 correspond to the steps 210-230 of the method described by reference to FIG. 2. In a further step 340, at least one number of count signals is counted in each detector element dependent upon the incident X-ray radiation. The steps 310, 320 which relate to a method for estimating a rate of randomly-occurring coincidences and a method for determining the coincidence of two X-ray signals acquired simultaneously can be carried out substantially simultaneously with the further step 340 in which in each detector element at least a number of count signals is counted dependent upon the incident X-ray radiation. In a further step 350, an X-ray image dataset is generated on the basis of the at least one number of count signals counted in each detector element and on the basis of the estimated real coincidence.

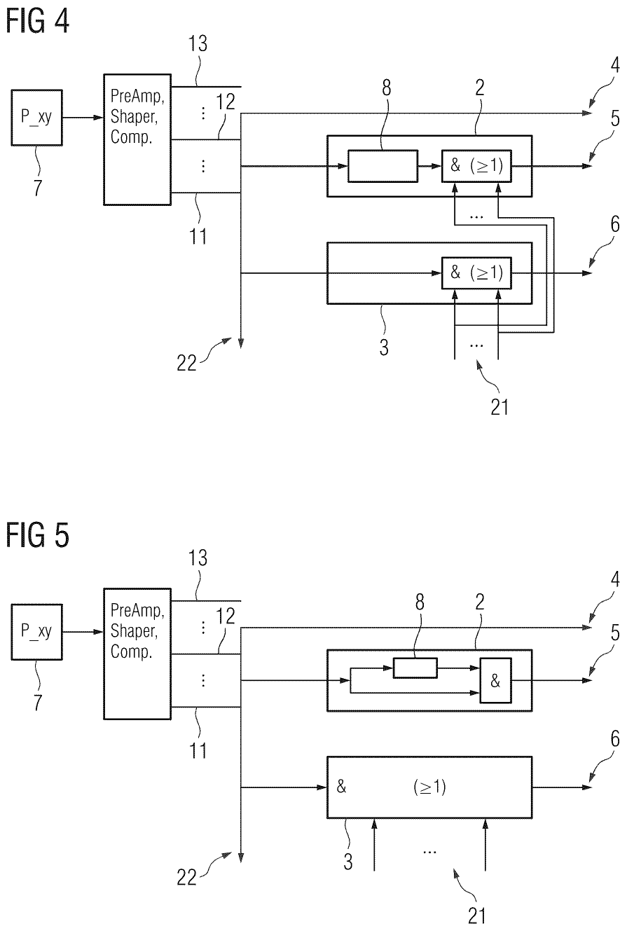

FIG. 4 shows a circuit example of a detector element according to one embodiment of the invention. The circuit comprises an input 7 in which electrical signals that have been converted from incident X-ray signals are received. The electrical signals are amplified and filtered with a plurality of comparators 11, 12, 13 which specify N different threshold values (threshold value 1, . . . threshold value i, . . . threshold value N). In this way it is possible to set what minimum energy registered X-ray signals must have in order to be counted. The exemplary circuit shows the circuit tree for one of the threshold values 12. Electrical signals are counted by a counter 4. It can occur herein that the entire energy of an X-ray quantum is not deposited in one detector element, but rather is distributed over two or more-typically adjacent-detector elements. This can be due to the fact that the charge clouds generated in the detector spread over more than one pixel, for example. In order to take account of this effect and to correct it, a coincidence unit 3 is provided. The coincidence unit 3 acquires cases in which an incoming electrical signal of the respective threshold 12 of the detector element is received in a specified timeframe simultaneously with an electrical signal from one or more adjacent detector elements. For this, both the electrical signal of this detector element and also electrical signals 21 of the adjacent detector elements are fed to the coincidence unit 3. A coincidence counter 6 then counts the number of the coincidence events in which at least one adjacent detector element exceeds a threshold simultaneously with the detector element under consideration. However, since the coincidence counter 6 is incremented not only in the case of real coincidences but also when a second, independent X-ray quantum deposits its energy in one of the adjacent detector elements simultaneously with the X-ray quantum incident in the detector element under consideration, an over-correction can occur, since more real coincidences are counted than actually occur. In accordance with this embodiment of the invention, a further coincidence unit 2 is provided for randomly-occurring coincidences (referred to below as the random coincidence unit 2). In this embodiment, further coincidences are counted with the random coincidence unit 2, specifically between a delayed signal of the detector element and a signal 21 from adjacent detector elements. Accordingly, signals 22 are fed from this detector element to a random coincidence unit 2 of other detector elements. The delay is achieved in this case by way of a delay 8 integrated into the random coincidence unit 2, which is arranged upstream of the actual acquisition of coincidences and incoming signals are temporally offset by a defined time interval. The time interval of the delay is therein selected such that the delay exceeds the maximum transit time differences of both the analogue and the digital signals in the circuit. It is thereby ensured that real coincidences can never increment this random coincidence counter 5. Therefore, only randomly-occurring coincidences are counted with the random coincidence counter 5. Since the temporal sequence of signals of a plurality of X-ray quanta is uncorrelated, the counting rate of the randomly-occurring coincidences does not change if a signal path is delayed, provided this delay is short in comparison with the sampling in a CT device. Effectively, in this embodiment, the digital coincidence circuit for each detector element is substantially doubled so that for each detector element, two coincidences are counted. The first coincidence is counted with the coincidence unit 3 and the coincidence counter 6 and acquires coincidences from the signal of the detector element and that of the neighbors, respectively without delay. This value represents the sum of real and randomly-occurring coincidences. For the second signal path, the digital signal of the detector element is delayed and coincidences with the adjacent detector elements are acquired with the random coincidence unit 2 and the random coincidence counter 5. Alternatively, for example, the signal 21 of the adjacent detector elements could also be delayed. This count value of the random coincidence counter 5 represents, in particular, the number of the randomly-occurring coincidences. Subsequently, the number of the randomly-occurring coincidences, acquired by the random coincidence counter 5, can be subtracted from the number of total coincidences, acquired by the coincidence counter 6, so that the number of real coincidences is obtained. As described here, for the two coincidence units 2, 3, the same number of signal inputs can be provided for electrical signals the coincidences of which are counted.

For example, in order to minimize the circuit complexity, the circuit can be simplified in that the coincidence tree for the randomly-occurring coincidences is constructed only partially. Accordingly, it can optionally be provided that not all the adjacent detector elements are called upon for the measurement of the randomly-occurring coincidences, but rather only a selection or even just a single one. In this optional variant, in order to correct the count value for the real coincidences, the count value of random coincidences can be scaled with the ratio of the number of adjacent detector elements for the uncorrected coincidences of the coincidence unit 3. For the coincidence unit 3 also, it can optionally be provided that signals are not called upon from all the adjacent detector elements. For example, the neighbors at the corners typically contribute little to the real coincidences and it can therefore be suitable to dispense with these. At the edges of a detector, it can also be useful to restrict the circuit to fewer inputs.

In the description of the exemplary embodiments below, it is above all the differences between the respective exemplary embodiments that are considered. In particular, commonalities are not described again in relation to each figure.

FIG. 5 shows a circuit example of a detector element according to a further embodiment of the invention. In this embodiment, the random coincidence unit 2 itself correlates the electrical signal of a detector element with a delayed copy of this signal. The value can also be scaled here with the number of detector elements the signals of which are used for the coincidence unit, in order to monitor a suitable correction of the count values of the coincidence counter 6. Optionally, it can be provided that the circuit for measuring the randomly-occurring coincidences is constructed only for a portion of the detector elements, for example in each case, for one or two detector elements in an N×M subgroup of detector elements, wherein N and M preferably take values of between 2 and 10. Thus, the circuit complexity can advantageously be further reduced.

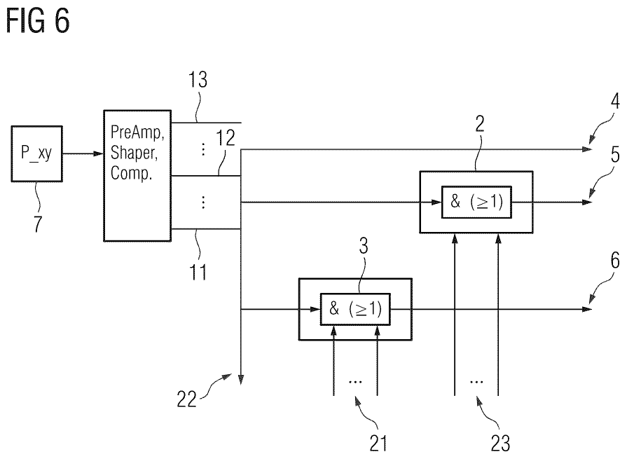

FIG. 6 shows a circuit example of a detector element according to a further embodiment of the invention. In this embodiment, randomly-occurring coincidences are acquired via a correlation of spatially separate detector elements. Therein, in the random coincidence unit 2, the coincidence of the detector element with the signals 23 of the next-but-one or even further-removed detector elements is counted. Since the detector elements are then spatially so distinctly separated that real coincidences practically cannot occur, herein only randomly-occurring coincidences are counted. Advantageously, for this, no additional delay 8 has to be built in, which is often relatively complex. For example, in this embodiment, the coincidence circuit for each detector element can also be doubled, so that the same number of signals arrive at the coincidence unit 3 as at the random coincidence unit 2. In the coincidence unit 3, for the measurement of the coincidences, in this embodiment also, signals 21 arrive from the directly adjacent detector elements, whereby all types of coincidences (random and real coincidences) are measured. Through the determination of the randomly-occurring coincidences by way of the random coincidence counter 5, the real coincidences can also be calculated out herein. In the embodiment shown in FIG. 6, optionally for the two coincidence units 2, 3, the same number of signal inputs can be provided for electrical signals the coincidences of which are counted.

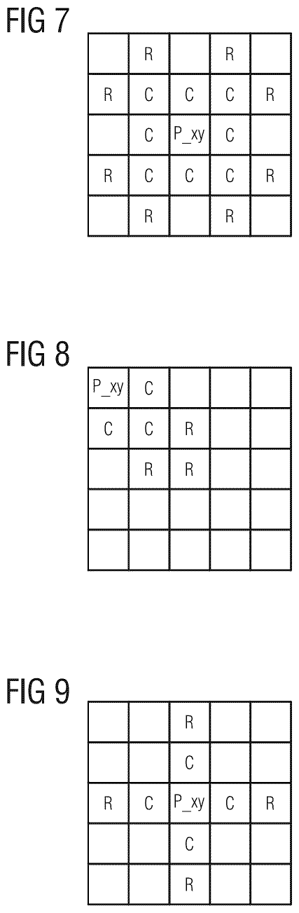

FIGS. 7-9 show a variant of the embodiment shown in FIG. 6 with regard to possibilities for the selection of the detector elements that are called upon for determining the coincidences. What is shown in each case is a subgroup of detector elements, wherein the detector elements are represented by the individual squares. P_xy stands therein for the detector element the real coincidence of which is to be determined. Identified by the letter C are detector elements the signals 21 from which are fed into the coincidence unit 3 for determining an uncorrected coincidence. Identified by the letter R are detector elements the signals 23 from which are fed into the random coincidence unit 2 for determining a randomly-occurring coincidence. Naturally, other configurations are conceivable in the context of the embodiment of FIG. 6. Different variants can also be mixed within a system, for example, for edges and corners of a detector portion. In the variant of FIG. 7, all the adjacent detector elements C for determining an (uncorrected) coincidence are called upon. In addition, the same number of detector elements is called upon, however in the form of next-but-one neighbors R for determining the randomly-occurring coincidence. In this example, all the next-but-one neighbors are called upon which, in a rectangle of next-but-one neighbors, are arranged not in the corners and not centrally on the sides of the rectangle. In FIG. 8, the detector element P_xy the real coincidence of which is to be determined is situated in a corner of the subgroup of detector elements. Accordingly, it has 3 neighbors C which are used for determining the (uncorrected) coincidence. For the determination of the randomly-occurring coincidence, accordingly, 3 next-but-one neighbors R are called upon. In this example, the 3 next-but-one neighbors are used which are not placed at the edge of the subgroup. In the embodiment according to FIG. 9, in each case, 4 closest neighbors C are called upon for determining the (uncorrected) coincidence and 4 next-but-one neighbors R are called upon for determining the randomly-occurring coincidence. Therein, only neighbors C and/or next-but-one neighbors R are called upon which lie in the rectangular subgroup of detector elements on the same horizontal or vertical line as the detector element P_xy, the coincidence of which is being determined.

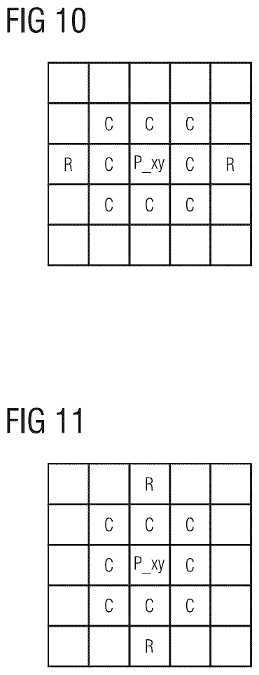

FIGS. 10 and 11 show further different variants with regard to possibilities for selecting the detector elements that are called upon for determining the coincidences. The naming of the detector elements corresponds to that in FIGS. 7 to 9. Therein, for the determination of the randomly-occurring coincidences, a smaller number of detector elements is used than for the determination of the (uncorrected) coincidences. In particular, only a small subgroup of the next-but-one detector elements is provided for determining the randomly-occurring coincidence. As also shown in the variant of FIG. 7, in the embodiments of FIGS. 10 and 11, all the adjacent detector elements C are called upon for determining the (uncorrected) coincidences. However, only 2 next-but-one neighbors R are called upon in each case for determining the randomly-occurring coincidences. In the embodiment shown in FIG. 10, the two next-but-one detector elements R in the X-direction are used. In the embodiment shown in FIG. 11, the two next-but-one detector elements R in the Y-direction are used. Preferably, in the use of one of these two variants, the measurement value of the randomly-occurring coincidences is scaled with a factor K/2, wherein K is the number of detector elements which is used in the coincidence unit 3. In this example, therefore, K would have the value 8.

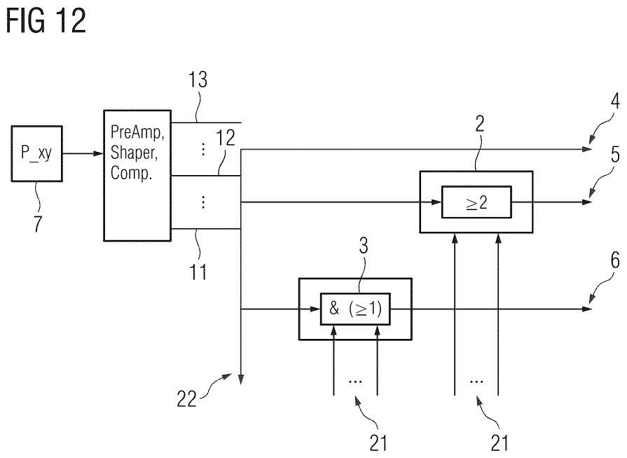

FIG. 12 shows a circuit example of a detector element according to a further embodiment of the invention. In this embodiment, the electrical signal of the detector element the rate of randomly-occurring coincidences of which is estimated, is itself not used for determining the randomly-occurring coincidence. The signals 21 which are fed to the random coincidence unit 2 originate from detector elements that are each adjacent to the detector element to be estimated. Signals 21 from adjacent detector elements can either enter only the coincidence unit 3 or only the random coincidence unit 2 or both. Detector elements the signals of which enter the random coincidence unit are not adjacent to one another.

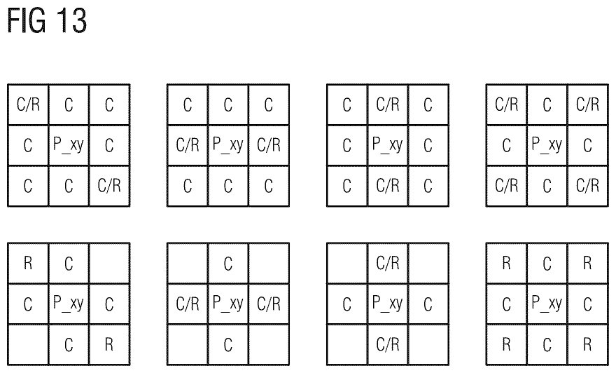

FIG. 13 shows different variants of the embodiment shown in FIG. 12 with regard to possibilities for selecting the detector elements that are called upon for determining the coincidences. The naming of the detector elements corresponds to that in FIGS. 7 to 11. In the variants, either the diagonals, the left and the right neighbor, the upper and lower neighbor or the 4 corners of the subgroup of detector elements shown are called upon for measuring the randomly-occurring coincidences. In the upper row, all the neighbors C, in the lower row, only the direct neighbors C in a cross form, i.e. not the oblique neighbors are used for the (uncorrected) coincidence unit 3. In principle, other combinations are also possible. It can optionally be provided to use a plurality of variants in one detector. Since a different number of detector elements contribute to the randomly-occurring coincidences than to the uncorrected coincidences, they can preferably be scaled accordingly before they are called upon for correction. The variants shown in FIGS. 12 and 13 have the advantage that the randomly-occurring coincidences of detector elements are derived which are spatially closer to the detector element to be estimated, so that spatial variations of the counting rates across the detector are less able to affect the value for the randomly-occurring coincidences. Particularly advantageous is the variant shown at bottom left since it represents the lowest circuit complexity with minimal loss of important information. In this variant, two next-but-one neighbors R which are arranged on a diagonal are called upon for measuring the randomly-occurring coincidences and only the closest neighbors C which are not the oblique neighbors are used for the (uncorrected) coincidence unit 3.

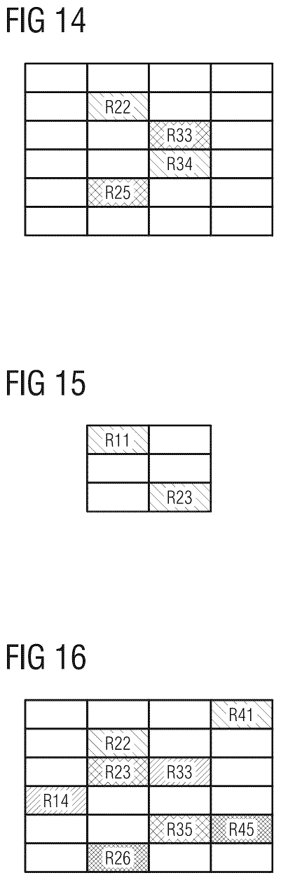

FIGS. 14 to 16 show further different variants with regard to possibilities for selecting the detector elements that are called upon for determining the coincidences. Each shows an M×N subgroup of detector elements. Therein, the circuit for measuring the randomly-occurring coincidences is not constructed for each detector element individually, rather for an M×N subgroup, only one detector element or a few detector elements are called upon for measuring the randomly-occurring coincidences. Thereby, the circuit complexity can be further reduced. In the variant shown in FIG. 14, for a subgroup of 4×6 detector elements, two separate random coincidence counters 5 are constructed for randomly-occurring coincidences and are representative for all 24 detector elements. The random coincidence units 2 of the random coincidence counters 5 each receive signals from two detector elements (R22 and R34 or R33 and R25). The sum of the two count values of the randomly-occurring coincidences can be scaled with the factor K/2 as a correction value, wherein K is the number of adjacent detector elements that is entered into the coincidence logic for the (uncorrected) coincidences. In the variant shown in FIG. 15, in a 2×3 subgroup, a random coincidence counter 5 is provided for all the detector elements of the subgroup, wherein the random coincidence units 2 of the random coincidence counter 5 each receive signals from two detector elements (R11 and R23). In the variant shown in FIG. 16, in a 4×6 subgroup, four random coincidence counters 5 are provided for all the detector elements of the subgroup, wherein the random coincidence units 2 of the random coincidence counters 5 each receive signals from two detector elements (R22 and R41 or R14 and R33 or R23 and R35 or R45 and R26).

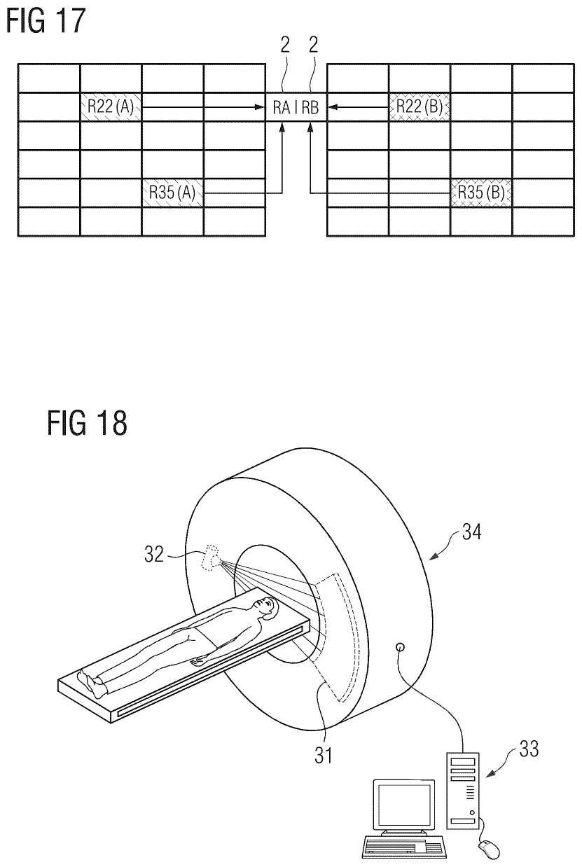

FIG. 17 shows a variant of the placement of the circuit for establishing the randomly-occurring coincidence according to one embodiment of the invention. The detector elements, represented here by rectangles, are subdivided into subgroups of detector elements of which two are shown here. In this embodiment, in each of two adjacent subgroups, two widely separated central detector elements are called upon for the establishing of the randomly-occurring coincidence. Intermediate spaces are present between the subgroups. The random coincidence units 2 of the two subgroups shown are arranged in the intermediate space between the subgroups. Accordingly, further random coincidence units 2 can be associated with further subgroups. If, in a circuit, for example, an ASIC, a plurality of 4×6 subgroups are constructed, it can be provided that the random coincidence units are provided alternatingly to left and right. The circuit is therein displaced out of the restricted space under the detector elements into the region between the subgroups of detector elements which might otherwise possibly be unused. Advantageously, the random coincidence circuit is therefore placed outside the actual matrix of detector elements. By this means, the detector elements can be configured identical and the entire space beside the detector elements is available for the circuits which must be present for all the detector elements. The subdivision and arrangement of the detector elements can also be different from that shown, for example, as in FIGS. 14 to 16.

FIG. 18 shows a computed tomography system according to one embodiment of the invention. The computed tomography system comprises a control module 33, an X-ray source 32 and a counting X-ray detector 31 with a large number of detector elements and an electrical circuit as described above, for example, as described in relation to FIGS. 4 to 17. The computed tomography system is configured to carry out a method as herein described, in relation, for example, to one of FIGS. 1 to 3. The X-ray source 32 and the X-ray detector 31 are arranged to be rotatable in a gantry 34.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible language), markup (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for estimating a rate of randomly-occurring coincidences in a counting X-ray detector, the X-ray detector comprising a number of detector elements, the method comprising:
acquiring X-ray signals by the X-ray detector and converting the X-ray signals into electrical signals at the detector elements;
passing on at least some of the electrical signals to signal inputs of a coincidence unit, the signal inputs including a first signal input and at least one further signal input,
wherein at least one of
signals for the first signal input are acquired in a first of the detector elements,
signals for the at least one further signal input are each acquired in another detector element not directly adjacent to the first of the detector elements, or
the signals for the at least one further signal input or the signals for the first signal input in an electrical circuit are temporally offset by a defined time interval before they are passed on to the coincidence unit;
counting coincidences of the signals passed on into the coincidence unit to determine at least one counting rate of acquired randomly-occurring coincidences; and
estimating a rate of randomly-occurring coincidences based on the at least one determined counting rate.

2. The method of claim 1, wherein
the signals for the at least one further signal input are each acquired in another of the detector elements, and
the signals for the at least one further signal input or the signals for the first signal input in the electrical circuit are temporally offset by a defined time interval before they are passed on to the coincidence unit.

3. The method of claim 1, wherein
the signals for the at least one further signal input are each also acquired in the first of the detector elements, and
the signals for the at least one further signal input or the signals for the first signal input in the electrical circuit are temporally offset by a defined time interval before they are passed on to the coincidence unit.

4. The method of claim 1, wherein
a plurality of coincidence units is provided, at least one coincidence unit is associated with each subgroup of detector elements,
the method is applied for each of the plurality of coincidence units, and
with each of the at least one coincidence unit, a rate of randomly-occurring coincidences is estimated for the detector elements of the associated subgroup.

5. The method of claim 1, wherein
the rate of randomly-occurring coincidences is estimated with the coincidence unit for a detector element to be estimated, the electrical signal of the detector element to be estimated is not fed into the coincidence unit, and
the signals for the first signal input and the signals for the at least one further signal input originate from detector elements that are each adjacent to the detector element to be estimated.

6. The method of claim 1, wherein
the detector elements are divided into subgroups of detector elements,
intermediate spaces are between the subgroups, and
the coincidence unit or a plurality of coincidence units is or are arranged in the intermediate spaces between the subgroups.

7. A method comprising:
performing the method of claim 1;
determining a coincidence of two X-ray signals acquired simultaneously according to a predetermined criterion on adjacent detector elements to establish uncorrected coincidences; and
correcting the uncorrected coincidences based on the rate of randomly-occurring coincidences to estimate a real coincidence.

8. The method of claim 7, wherein
a coincidence unit is provided for establishing the uncorrected coincidences and for estimating the randomly-occurring coincidences, and
for each of the coincidence unit for establishing the uncorrected coincidences and the coincidence unit for estimating the randomly-occurring coincidences, a same number of signal inputs are provided for electrical signals, the coincidences of which are counted.

9. The method of claim 8, wherein
for establishing the uncorrected coincidence and also for estimating the randomly-occurring coincidence, signals are provided for the signal inputs of the respective coincidence unit from the detector element, the real coincidence of which is to be determined, and from one or more of the adjacent detector elements, and
only a subgroup of the adjacent detector elements is provided for at least one of establishing the uncorrected coincidence or estimating the randomly-occurring coincidence.

10. The method of claim 8, wherein at least one of
only a subgroup of the adjacent detector elements is provided for establishing the uncorrected coincidence, or
only a subgroup of next-but-one detector elements is provided for estimating the randomly-occurring coincidence.

11. The method of claim 7, wherein
the real coincidence is transferred as a count value, and
at least one of the randomly-occurring coincidence or the uncorrected coincidence is also transferred as an additional count value.

12. The method of claim 7, wherein the at least one counting rate of the randomly-occurring coincidences is used to monitor a paralysis of another counter of signals.

13. A method for recording an X-ray image dataset of an object with an X-ray system, the method comprising:
counting at least one number of count signals based on incident X-ray radiation in each detector element;
performing the method of claim 7; and
generating the X-ray image dataset based on the at least one number of count signals counted in each detector element and the estimated real coincidence.

14. A counting X-ray detector comprising:
a number of detector elements; and
at least one electrical circuit with at least one coincidence unit, wherein the X-ray detector is configured to perform the method of claim 1.

15. A medical imaging device comprising:
a counting X-ray detector; and
a control module, wherein the medical imaging device is configured to perform the method of claim 1.

16. The method of claim 2, wherein the signals for the at least one further signal input are each acquired in a detector element directly adjacent to the first detector element.

17. The method of claim 4, wherein at least one coincidence unit is associated with a subgroup of spatially adjacently arranged detector elements.

18. The method of claim 7, wherein the determining includes counting a number of coincidence signals for each detector element with at least one adjacent detector element.

19. The method of claim 12, wherein the at least one counting rate of the randomly-occurring coincidences is used to correct the paralysis of the another counter of signals.
